(19) United States
(12) Patent Application Publication
(10) Pub. No.: US 2010/0249566 A1
(43) Pub. Date: Sep. 30, 2010

(54) Interactive device for monitoring and reporting glucose levels with integrated atomic clock module

(76) Inventors: Frank P. Suess, Wellington, FL (US); Oliver Suess, Wellington, FL (US); Steven Thuss, Wellington, FL (US)

Correspondence Address: Stein Law, P.C., 24 Woodbine Avenue, Suite 4, Northport, NY, 11768, US

(21) Appl. No.: 12/802,067

(22) Filed: May 28, 2010

Related U.S. Application Data
(63) Continuation-in-part of application No. 11/438,566, filed on May 22, 2006, now abandoned.
Application No. 11/438,566 is a continuation-in-part of application No. 12/049,749, filed on Mar. 17, 2008, now abandoned.

(51) Int. Cl. A61B 5/145

(52) U.S. Cl. 600/365

(57) ABSTRACT

An apparatus for determining the amount of glucose in a patient comprising a CPU for receipt and analysis of data; a glucose testing means for testing the amount of glucose in the patient's blood, providing patient data to the CPU, and determining the amount; a storage means for storing data linked to the patient; a display means for displaying the glucose amount and/or glucose data and interfacing with the patient; a voice processing means for processing the glucose amount and/or glucose data and synthesizing an auditory output and optionally includes further instructions as determined by a treating physician and the CPU after analysis of the glucose amount and/or glucose data; a data acquisition means for acquiring data; a digital storage means for storing data; and an atomic clock means for generating real time clock signals from time code signals transmitted by a time standard and received by an antenna.

### CONTINUING DATA

This is a continuation-in-part application of U.S. patent application Ser. No. 12/049,749, filed on Mar. 17, 2008 which is a continuation-in-part of U.S. patent application Ser. No. 11/438,566, filed on May 22, 2006. Both U.S. patent application Ser. No. 12/049,749 and U.S. patent application Ser. No. 11/438,566 are incorporated herein by reference.

### TECHNICAL FIELD

The present disclosure generally relates to the field of diabetes management, and more particularly to glucose meters for the self-monitoring of blood glucose wherein patient-relevant information associated with the disease state is flagged and stored with the patient's active data such that the patient and physician can have immediate access and render modifications and monitoring as necessary. In a preferred embodiment of the present invention, an atomic clock module is incorporated into the circuitry of the glucose meter for simplifying the setting of date and time and providing valid, objective time and date stamps.

### BACKGROUND

Any publications or references discussed herein are presented to describe the background of the invention and to provide additional detail regarding its practice. Nothing herein is to be construed as an admission that the inventor is not entitled to antedate such disclosure by virtue of prior invention.

The instant invention relates to a glucose meter which incorporates a means for the patient to record time specific data relevant to diabetes management and an atomic clock module to provide accurate time and date information in connection with such data, in order to either self-evaluate or have a physician evaluate the correlation between inputted data and the patient's recorded blood glucose level(s) over a period of time.

Diabetes mellitus, commonly referred to as diabetes, is a chronic disease in which an individual's blood glucose levels become abnormally high due to an inability to break down glucose. The hormone insulin is responsible for regulating glucose levels in the blood. Diabetics produce either a deficient amount of insulin to break down the glucose present in the blood, or are resistant to insulin and therefore cannot use it properly. An estimated 17 million persons in the United States have diabetes, with almost 1 million new cases being diagnosed each year.

Diabetes is known to cause damage to the small and large blood vessels, leads to diabetic blindness, kidney disease, amputations of limbs, stroke, and heart disease. According to the U.S. Centers for Disease Control and Prevention, more than 3 million Americans who have diabetes are visually impaired.

Self monitoring of one's blood glucose has been determined to be an effective tool to manage diabetes. Self monitoring of blood glucose is recommended by the FDA for all people with diabetes. It is recognized that self monitoring of blood glucose will allow the user to, among other things: (1) keep track of their glucose levels over time, (2) make day to day decisions for managing glucose, (3) recognize emergency situations; and (4) educate themselves on how to manage their blood glucose levels. Accordingly, a growing number of Americans suffering from diabetes monitor their blood glucose at home. It is understood by one of ordinary skill in the art that accurate time and date information must be recorded simultaneously with an individual's information (such as his or her glucose levels, for example) to be reliable for medical and diagnostic purposes.

Checking one's blood glucose level allows a physician and/or the individual to determine how much insulin should be taken to maintain normal blood glucose levels. The amount of insulin a diabetic requires will likely vary with age, a change in diet or lifestyle, stress, or illness; the amount of insulin required to maintain normal blood glucose levels will thus likely vary (to varying degrees) from day to day. It is therefore necessary for a diabetic to periodically check his or her glucose levels to ensure they are taking the proper amount of insulin and to determine whether changes to diet, exercise and the like are sufficient/appropriate to improve management and longevity.

It is recommended that most diabetics monitor their blood glucose levels more than one time per day to preempt a major episode caused by low blood glucose levels (hypoglycemia). Many diabetes management plans direct the individual to test glucose three or more times a day under normal conditions, and more frequently during times of illness or stress. Typically diabetics are directed to check their blood glucose levels before and after meals or exercise, at bedtime, and any other time they experience signs or symptoms of hyperglycemia or hypoglycemia. However, the specific times and frequency a diabetic should conduct such checks are typically determined by the individual's physician or by the happening of an event, rather than correlating to normal life styles, like ingestion times, quantities and types of food, exercise, stress, illness and the like.

Many factors can cause one's blood glucose levels to fluctuate such as the amount of insulin taken, the amount of food ingested (or not ingested), the type of food ingested, and the amount one exercises. As such it is preferable for a diabetic to keep records of not only periodic blood glucose levels, but information regarding diet and exercise and to report this information to the treating physician to determine proper medical intervention. These records, over time, provide practical data regarding causes for fluctuations in an individual's blood glucose levels, and hence also instruct one how to predict when the individual's blood glucose levels will fluctuate above or beyond normal levels in response to a particular activity or event and how to counter the same. Glucose meters measure the amount of glucose present in an individual's blood.

To use a glucose meter, the user typically places a small sample of blood on a test strip. A chemical present on the test strip (typically glucose oxidase, dehydrogenase, or hexokinase) then combines with the blood to create a reaction. When the test strip is inserted in to a glucose meter, the meter measures the chemical reaction and translates it into a score indicating the individual's blood glucose level. The score is often displayed or printed. Glucose meters have also been developed which measure the presence of glucose by measuring the amount of electricity that can pass through a sample of blood, or how much light reflects from the sample, but these are complex and generally less reliable in testing actual blood glucose levels.

To keep a history of the user's blood glucose testing, glucose meters often require the user to set the date and time. As such, an individual's glucose levels can be recorded over time, and analyzed to determine the proper protocol for a given individual based on their past history. To record a user's glucose levels over time, many glucose meters require that the user set the date and time prior to each use and every use, to differentiate one blood test from another. In that many diabetics test their blood glucose levels multiple times a day, blood glucose meters which require that the user set the date and time prior to each use can quickly become extremely frustrating to use. Furthermore, because most blood glucose meters rely on the user to input the time and date information, such information is subject to user error, and could therefore make an individual's past history, as recorded by the meter, unreliable. Indeed, to properly interpret glucose trends with time, it is critical that correct date and time information be recorded with every glucose test/measurement. Heretofore unknown is a blood glucose meter which is able to reliably set time and date information itself upon powering up, saving the user the task of manually entering time and date information.

Among the problems associated with the self monitoring of blood glucose levels are the ability to associate a given score from a glucose meter with the diet and activities (and remainder of an individual's regimen). It has heretofore been left to the individual to manually record data such as when they ate, what they ate, when they exercised, how long they exercised, and the like typically after an event by memory to the physician to understand the correlation with such activity and the individual's blood glucose levels. This too creates the potential for error, as a user is essentially required to carry around a notebook or the like to record relevant information simultaneously with time and date information.

Virtually heretofore unknown was the ability or desire to maintain such records in the absence of a triggering event that would require someone to do perform the same. Indeed, in the absence of a triggering event, an individual is unlikely to perform the laborious task of manually recording information regarding daily aspects of his or her diet or lifestyle, stress, or illness, for example.

It is recognized that an understanding of said correlation will allow the physician and/or patient to accurately determine the proper amount of insulin needed, or whether a simple change in lifestyle would suffice to bring the individual's blood glucose level within a normal range. Thus, it is an object of the instant invention to provide a glucose meter which allows the user to record information relating to their diet, amount of exercise, level of stress or illness and other circumstances concurrently with the user's periodic testing and recording of their blood glucose level.

In one embodiment of the present invention, the user can record information relating to their diet, amount of exercise, level of stress or illness and other circumstances by inputting information via a keypad or keyboard, for example. It is also envisioned that the user can record information relating to their diet, amount of exercise, level of stress or illness and other circumstances via a voice recorder, for example. It should be understood, however, that the user may record information relating to their diet, amount of exercise, level of stress or illness and other circumstances by any means known in the art to record information to an electronic device.

Known in the art are glucose meters which include an audio output to aid a user with vision loss in self-monitoring blood glucose levels. One such device is commercially available from Roche Diagnostics under the trade name “Accu Chek Voicemate.” This single unit device provides both audio instruction and audible test results. Also commercially available are speech synthesizers which attach to glucose meters to similarly provide both audio instruction and audible test results. Examples of such commercially available voice synthesizers are the “Voice Touch” speech synthesizers produced by Myna Corporation for use with LifeScan glucose meters, and “Digi Voice” speech synthesizers produced by Science Products also for use with certain LifeScan glucose meters. Such meters, however, fail to provide any means for a user to record information relating to his or her personal circumstances concurrently with the user's periodic testing and recording of their blood glucose level and/or any means to simplify the setting of time and date by reliably set time and date information itself upon powering up.

Another problem associated with the self monitoring of blood glucose levels, discussed briefly above, is that blood glucose meters known in the art require that the user set the date and time at least once. This creates the possibility for user error, as the user can enter incorrect time and/or date information for a variety of reasons, or fail to enter such information altogether. For example, the user may forget to input correct date and time information after purchasing the meter, power failure, traveling to a different time zone, or daylight savings time, etc. Additionally, the user may simply enter time and date information incorrectly, falsely believing that such information is correct. It is recognized that the more a user is required to enter date and/or time information, the more likely he or she is enter incorrect information. If the date and time information entered into a glucose meter is not accurately recorded each and every time the meter is used, the history recorded by the meter will become inaccurate and hence be of little value. Even if set correctly, blood glucose meters known in the art which include a timekeeping device, such as a clock, employ traditional mechanical and/or electronic clocks which utilize which are inaccurate and are likely to fail over time. Heretofore unknown is a glucose meter which utilizes a reliable timekeeping mechanism which sets time and date information itself upon powering up so as to ensure information relating to a user's glucose trends (as determined by blood glucose testing over time), diet, amount of exercise, level of stress or illness and other circumstances is entered with an accurate time and date (such as through time and date stamping, for example) which correlates to the time and date such information was recorded and/or inputted.

Thus, it is also an object of the instant invention to provide a glucose meter which simplifies the setting of date and time and provides valid, objective time and date stamps by incorporating an atomic clock and/or radio clock module into the glucose meter. This ultimately adds value by minimizing patient set up time and helping increase patient compliance, permitting the capture of relevant data with objective date and time stamping accurately showing frequency of use and results, as well as providing other data, for medical and diagnostic purposes, with a reliably valid timestamp.

Many attempts have been made to provide a device for the self-monitoring of blood glucose levels which is capable of recording accurate time and date information. U.S. patent application Ser. No. 11/851,194 by Estes et al. describes medical devices, including glucose meters, which receive an external reference signal from a controller independent of the device to provide automatic time-setting. U.S. patent application Ser. Nos. 12/031,660 and 12/031,664 by Galasso et al. describe an infusion pump which implements modifications associated with blood glucose monitoring based on time and date information. U.S. patent application Ser. No. 11/496,606 by Goldsmith et al. describes a watch controller which includes time-telling functions and communicates with an infusion pump and/or glucose sensor independent from the watch controller. U.S. patent application Ser. No. 11/704,526 by Ray et al. describes a method of validating date and time information on a blood glucose meter in which time is checked for accuracy via an atomic clock module over a computer network. U.S. patent application Ser. Nos. 10/741,967 and 10/770,946 by McMahon describe a system for diabetes management in which time and location information is obtained by a server with a plurality of networks.

Heretofore unknown is a portable glucose meter which allows the user to record information relating to their diet, amount of exercise, level of stress or illness and other circumstances concurrently with the user's periodic testing and recording of their blood glucose level which incorporates an atomic clock module into the circuitry of the glucose meter, for simplifying the setting of date and time and providing valid, objective time and date stamps. Therefore, it would be desirable to provide an apparatus that provides such advantages.

### SUMMARY OF THE INVENTION

Accordingly, the present disclosure provides a device for determining glucose levels in a patient having a CPU for receipt and analysis of data; a glucose testing means for testing the amount of glucose in the patient's blood at the point of the test, providing data related to the glucose amount to the CPU, and the CPU for determining the amount; a storage means for storing the glucose amount and/or other glucose data linked to the patient; a visual display means for displaying the glucose amount and/or glucose data and interfacing with the patient; a voice processing means for processing the glucose amount and/or glucose data and synthesizing an auditory output that conforms with the glucose amount and/or glucose data and optionally includes further instructions as determined by a treating physician and the CPU after analysis of the glucose amount and/or glucose data; a data acquisition means for acquiring the following data concurrently with the glucose amount then tested: (1) dietary consumption; (2) exercise; (3) medical information (size, BMI, other conditions, other medications, etc.); and (4) comments; (g) a digital storage means for storing the data acquired by the data acquisition means, the glucose amount and/or other glucose data for later data retrieval; and atomic clock means for generating real time clock signals from time code signals transmitted by a time standard and received by an antenna.

The present disclosure also provides for an “all in one” integrated device for determining glucose levels in a patient comprising: a CPU for receipt and analysis of data; a glucose testing means for testing the amount of glucose in the patient at the point of the test, providing that test data to the CPU, and the CPU for determining a level of glucose; a visual display means for displaying glucose levels and interfacing with the patient; a voice processing means for processing the glucose level and synthesizing an auditory output that conforms with the glucose level, wherein said voice processing means is bidirectional and receives additional data from the patient and via a keypad concurrently with the glucose level then tested, including: dietary consumption; exercise; medical information (size, BMI, other conditions, other medications); and comments; a storage means for storing said test data, said glucose level and said additional data linked to the patient for later retrieval; an input/output means for transmitting said test data, said glucose level and said additional data to a treating physician and receiving further instructions as determined by the treating physician after the determination of said glucose level; and an atomic clock means for generating real time clock signals from time code signals transmitted by a time standard and received by an antenna.

In one particular embodiment of the present invention, time and date information is automatically set upon powering up the glucose meter by obtaining accurate time and date information via an atomic clock means. In another embodiment of the present invention, the user is required to input information relating to the time zone in which the individual is located, which the atomic clock means will use to obtain accurate time and date information for that location. In this embodiment, a simple drop down menu or other means is employed for setting the time zone (as an offset to the broadcast time from an atomic clock) and is the only input required to accurately set time and date information.

Information relating to an individual's diet, amount of exercise, level of stress or illness and other circumstances concurrently with the individual's periodic testing and recording of their blood glucose level may be time and date stamped to accurately record the time and date such information was recorded/inputted to provide relevant data with objective data and time and date stamping to accurately show the frequency of use and results, as well as provide other data, for medical and diagnostic purposes, with a reliably valid timestamp.

In accordance with the teachings of the instant invention, an interactive glucose meter (a “glucose testing means”) is disclosed which includes an input/output to the physician, by which the physician can evaluate the recorded blood glucose results and other information inputted by the patient for analysis, thereby eliminating the need for the patient to schedule an appointment to meet with the physician in person. The physician's instructions are provided and output via the voice processor and/or the visual display. Accuracy of time and date of recorded blood glucose results and other information inputted by the patient is ensured by the atomic clock means/module which provides reliable time and date stamps for all information recorded/inputted.

An atomic clock is a timekeeping device that uses an atomic resonance frequency standard as its timekeeping element. Atomic clocks are the most accurate timekeeping devices, and are currently being used by international time distribution services to record time, which is transmitted by a radio transmitter. Radio clocks are synchronized by a time code bit stream transmitted by a radio transmitter connected to a time standard such as an atomic clock. As used herein “atomic clock” or “atomic clock means” is meant to include radio clocks.

In accordance with the teachings of the present invention, a glucose meter is provided which allows the user to record information relating to their diet, amount of exercise, level of stress or illness and other circumstances concurrently with the user's periodic testing and recording of their blood glucose level so as to flag and store the data with the patient's active data such that the patient and physician can have immediate access and render modifications and monitoring as necessary. Said flagging will allow the physician or individual to better understand the correlation between the patient's lifestyle and his or her blood glucose level. The user may input the desired information via a keypad or keyboard which may be incorporated with the glucose meter or attached to the glucose meter's data port. The atomic clock module/means will provide a time and date stamp for each flagged event, thus providing relevant chronological data which a physician, the individual, or a member of the individuals diabetes management team may use to determine the proper manner to deal with an individual's glucose trends, whether by modifying the amount of insulin provided or simply changing exercise or dietary habits.

Alternatively, information may be recorded via a recording device incorporated directly into the glucose meter or attached to the glucose meter's data port. The voice recordings can be recorded in *.wav or other format. As with other information discussed above, the voice recordings and information entered by keypad or keyboard are provided time and date stamps via the atomic clock means of the present invention, to accurately record the time and date such information was entered to provide a reliable history log. It will be understood by one of ordinary skill in the art that the voice recording device incorporated into the glucose meter of the present invention may include any device capable of recording audible sound.

In accordance with the teachings of the present invention, a glucose meter is provided which incorporates a voice processor/synthesizer to record/process spoken data (or any other audible sound) and speak information relating to such date, such as instructions and results, to the user aloud. The synthesizer's voice can be either male or female, and may be translated into a multiplicity of languages without deviating from the spirit of the instant invention. The volume of the voice synthesizer may increased or reduced depending on the user's preference. The voice synthesizer may alternatively be reduced to zero (“muted”), should the user desire silence, in which case the instructions and/or results may be viewed directly on the display screen. It will be understood by one of ordinary skill in the art that the voice processor/synthesizer incorporated into the glucose meter of the present invention may include any device capable of recording/processing data relating to an audible sound and/or speaking such data aloud.

In accordance with the teachings of the present invention, a glucose meter is provided which incorporates a large display screen to aid the visually impaired or for people who are assisting them. The display screen functions independently of the voice processor/synthesizer so as to allow the user to have the instructions and/or results presented via the display screen and/or the voice synthesizer. In a preferred embodiment, the atomic clock means of the present invention provides the correct time and date which is displayed on the display screen.

The various features of novelty which characterize the present invention are expressly and unambiguously delineated in the claims annexed to and forming part of the disclosure. For a better understanding of the present invention, its practical advantages, and specific objects attained by its use, reference should be had to the drawings and descriptive matter in which there are illustrated and described preferred embodiments of the invention.

Other features will become apparent from reading the disclosure and claims of the instant invention.

### BRIEF DESCRIPTION OF THE DRAWINGS

The features of the present disclosure will become more readily apparent from the specific description accompanied by the following drawings, in which:

FIG. 1 is a topographical overview of a preferred embodiment of the present invention in accordance with the principles of the present disclosure;

FIG. 2 is a component diagram of the various hardware components of the preferred embodiment in accordance with the principles of the present disclosure; and

FIG. 3 is a flow chart of patient entry showing the interface between the various components of a preferred embodiment of the present invention in accordance with the principles of the present disclosure.

Like reference numerals indicate similar parts throughout the figures.

### DETAILED DESCRIPTION OF THE INVENTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

Also, as used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes'from the one particular value and/or to the other particular value. Similarly, when values are expressed as approximations, by use of the antecedent “about,” it will be understood that the particular value forms another embodiment.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non claimed element as essential to the practice of the invention.

All publications, patents and patent applications cited in this specification are herein incorporated by reference in their entirety as if each individual publication, patent or patent application were specifically and individually indicated to be incorporated by reference.

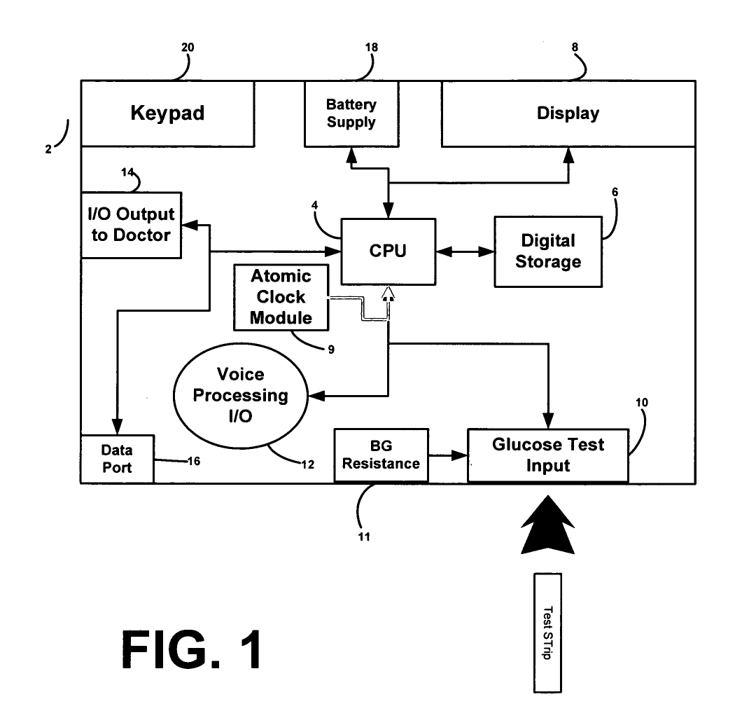

The following discussion includes a description of the apparatus of the subject invention, and related components. Alternate embodiments are also disclosed. Reference will now be made in detail to an exemplary embodiment of the present disclosure, which is illustrated in the accompanying figures. Turning now to FIGS. 1-3, the components of an apparatus 10 in accordance with the principles of the present disclosure, are illustrated.

As shown in FIG. 1, at the heart of the apparatus/device is CPU 4 which engages digital storage 6. It should be recognized that the database, or digital storage 6 is such that each individual user has, associated with a tag specific to the individual, one or more of the following items:

(a) glucose levels;

(b) dietary consumption;

(c) exercise;

(d) medical information (size, BMI, other conditions, other medications, etc.); and

(e) comments.

The information indicated to be stored per patient in digital storage 6 and tagged to the individual patient can be maintained for long periods of time, depending upon the size of storage device 6. Likewise, the information can be downloaded from storage device through data port 16 to any other device. It should be appreciated that this information can be hard wired through port 16, or WI FI communicated, or any of a number of different mechanisms known or hereinafter developed for the transmission of digital data.

Information can be added to database 6 in a plurality of forms. For example, voice processing I/O 12 allows the patient to enter data by speaking into the monitor 2 and the information is typically digitized and stored (as in a *.wav) file tagged to that patient in digital storage (a/k/a database) 6. Likewise, the individual/patient can enter data via keypad 20. Information is displayed via display 8, or can be spoken back to patient via voice processing I/O 12.

All information added to database 6 is provided with a time and date stamp by atomic clock module 9. In one embodiment of the present invention, database 6 stores information regarding monitor 2, such as the manufacturer, date of manufacture, model number, serial number, and the like for future retrieval. In one particular embodiment of the present invention, information regarding monitor 2 is displayed by a display means and/or spoken aloud when monitor 2 is turned on. As such, it is recognized that database 6 may store information related to an individual and/or monitor 2. All information can be extracted from monitor 2 at any time and can be used to analyze an individual's diabetes management strategy by reviewing their blood glucose levels over time.

It will be appreciated by one of ordinary skill in the art that atomic clock module 9 supplies real time signals to CPU 4. Atomic clock module 9 is synchronized by a time code bit stream transmitted by a radio transmitter connected to a time standard such as National Institute of Standards and Technology in Fort Collins, Colo. or any other known international time standard broadcasts. Atomic clock module 9 includes an antenna 22 for receiving the time code and contains components known in the art to convert the time code into a digital time code and decoding the time code bit stream to a form usable by CPU 4. In particular antenna 22 receives a radio frequency time code signal, which is converted into a digital time and date code signal via CPU 4. CPU 4, therefore decodes the digital signal which is then output to any of the various components of monitor 2 including for example, the glucose testing means (glucose test strip input 10 and/or BG Resistance 11), display 8, voice processing I/O 12, and I/O Output to Doctor 14 to provide accurate time and date information which can then be used to create time and date stamps in connection with information inputted and/or recorded into monitor 2.

In one embodiment of the present invention, monitor 2 is synchronized when monitor 2 is turned on, automatically, without the use of any key or button. The individual is not required to manually set the date and time, thus avoiding the potentials for user error discussed above. In one embodiment of the present invention, the user is required to input information relating to the time zone in which the individual is located, which atomic clock means 9 will use to obtain accurate time and date information for that location. In this embodiment, a simple drop down menu or other means is employed for setting the time zone (as an offset to the broadcast time from an atomic clock) and is the only input required to accurately set time and date information.

Atomic clock module 9, in stark contrast to what is known in the art, is synchronized by a time code bit stream transmitted by a radio transmitter connected to a time standard. Accordingly, atomic clock module 9 does not require a connection (be it wireless or wired) to a network, such as a computer network. Atomic clock module 9, which may be powered by batter supply 18 or any other power means, includes an antenna 22 for receiving the time code and contains components known in the art to convert the time code into a digital time code and decoding the time code bit stream to a form usable by CPU 4. By allowing atomic clock module 9 to connect to a time standard via a radio transmitter to synchronize time and date information, atomic clock module 9 is operational whenever monitor 2 is turned on, and does not require a connection to a computer or any other networking or sharing device. As such, glucose monitors and other devices for testing and/or maintaining blood glucose levels which employ an atomic clock module or a radio clock module over a computer network (such as U.S. patent application Ser. No. 11/704,526 by Ray et al. and U.S. patent application Ser. Nos. 10/741,967 and 10/770,946 by McMahon, discussed hereinabove) are easily distinguishable from the present invention.

Furthermore, also in contrast to what is known in the art, atomic clock module 9 is incorporated into the circuitry of monitor 2, and is not provided as a distinct structural element. Indeed, glucose monitors and other devices for testing and/or maintaining blood glucose levels which employ an atomic clock (such as U.S. patent application Ser. No. 11/851,194 by Estes et al. and U.S. patent application Ser. No. 11/496,606 by Goldsmith et al., also discussed hereinabove) describe medical devices, including glucose meters, which receive an external reference signal from a controller independent of the device to provide automatic time-setting. It is understood that incorporating atomic clock module 9 into the circuitry of monitor 2 will avoid many of the shortcomings related to glucose monitors which utilize separate elements/components for time-setting and glucose testing, such as a failure for each element/component to communicate with one another.

In one particular embodiment of the present invention, when monitor 2 is turned on, a drop down menu is displayed on display 8 for setting the time zone (as an offset to the broadcast time). No other input is required to set the date and time of monitor 2. In another embodiment of the present invention, the time zone may be set orally via voice processing I/O 12. It will be understood by one of ordinary skill in the art that voice processing means I/O 12 may include any means capable of processing digital and/or electronic data and synthesizing an auditory output that conforms with the same.

Critical to the device is an “all in one” aspect in which glucose quantities are determined typically by insertion of a glucose strip that has been impregnated with a sample of that patient's blood, and inserted via quantity test, the test is analyzed and output to element 10, and back to CPU 4 for storage in database 6. The glucose reading is tagged to all other information that is stored, as heretofore indicated per patient at specific dates and times such that a physician and/or patient can see all such information or hear all such information stored over a period of time. The date and time such information is provided is timestamped by atomic clock module 9, thus ensuring the accuracy of such information.

I/O output to doctor 14 can occur in the ordinary course of computer science, or can be downloaded via data port 16 and linked to the physician. Also shown is replaceable battery 18, which can be nickel cadmium (for rechargeability) or lithium (for size) or any of a number of other power supplies. Indeed, the device can be plugged into an AC outlet, provided that a transformer is included to ensure proper power management. As such, replaceable battery 18 may include any D.C. power unit known in the art to supply electrical power to the various components of device 2.

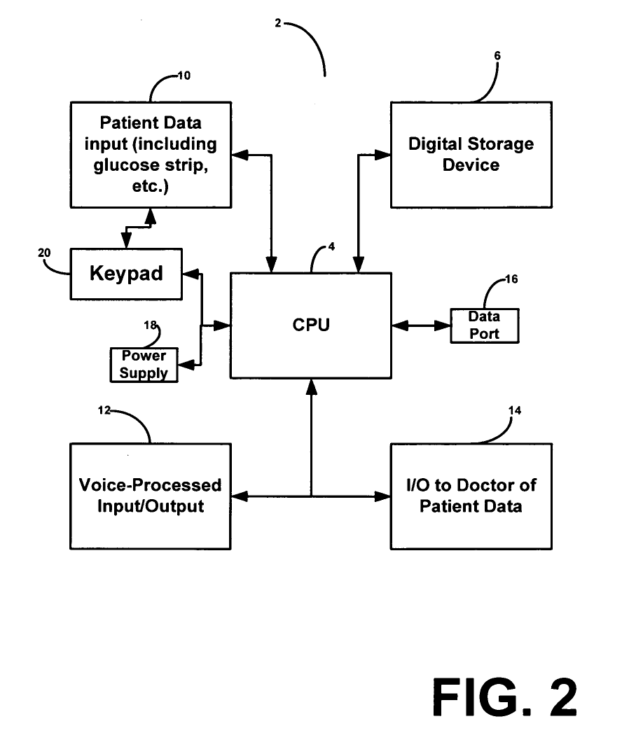

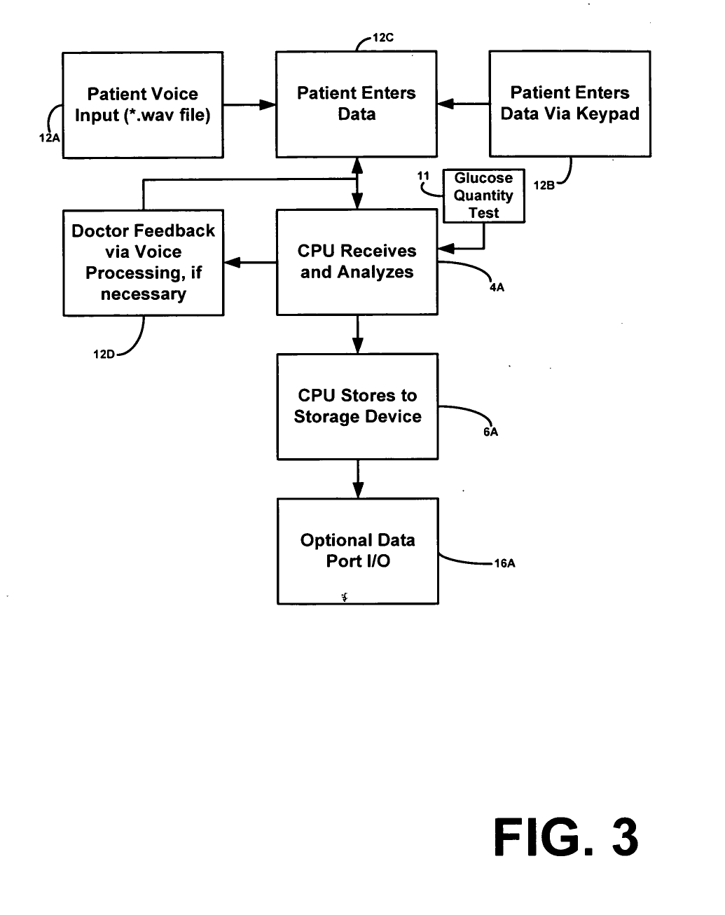

FIG. 2 also shows device 2, in a component blow up model, in which like numbered items have the same function as heretofore indicated. FIG. 3 shows a flow chart wherein patient enters data at step 12C (which can be via keypad 20 if the patient so elects, via step 12B. Also, the patient can engage the voice I/O processor 12 which can not only ask questions and receive answers, but also can simply record the information that the patient provides (including that indicated hereinabove). After (or concurrently) with the input of patient data is glucose quantity test 11 which outputs results to CPU 4A for analysis. Shown also is doctor feedback 12D via voice processing if necessary, or other means (preferably voice interface). When the information is collected it is stored to the storage device via step 6A and output, optionally via data port I/O 16 A.

It will be understood that various modifications may be made to the embodiments disclosed herein. Therefore, the above description should not be construed as limiting, but merely as exemplification of the various embodiments. Those skilled in the art will envision other modifications within the scope and spirit

I claim:

1. A device for determining the amount of glucose in a patient comprising:
(a) a CPU for receipt and analysis of data;
(b) glucose testing means for testing the amount of glucose in the patient's blood at the point of the test, providing data related to the glucose amount to the CPU, and the CPU for determining the amount;
(c) storage means for storing the glucose amount and/or other glucose data linked to the patient;
(d) visual display means for displaying the glucose amount and/or glucose data and interfacing with the patient;
(e) voice processing means for processing the glucose amount and/or glucose data and synthesizing an auditory output that conforms with the glucose amount and/or glucose data and optionally includes further instructions as determined by a treating physician and the CPU after analysis of the glucose amount and/or glucose data;
(f) data acquisition means for acquiring the following data concurrently with the glucose amount then tested:
(1) dietary consumption;
(2) exercise;
(3) medical information (size, BMI, other conditions, other medications, etc.); and
(4) comments;
(g) digital storage means for storing the data acquired by the data acquisition means, the glucose amount and/or other glucose data for later data retrieval; and
(h) atomic clock means for generating real time clock signals from time code signals transmitted by a time standard and received by an antenna.

2. The device of claim 1, wherein said data retrieval is achieved via an input/output to the physician.

3. The device of claim 1, wherein physician's instructions are provided and output via said voice processing means.

4. The device of claim 1, wherein said the acquisition means acquires data via the voice processing means and/or a keypad.

5. An “all in one” integrated device for determining glucose levels in a patient comprising:
(a) a CPU for receipt and analysis of data;
(b) glucose testing means for testing the amount of glucose in the patient at the point of the test, providing that test data to the CPU, and the CPU for determining a level of glucose;
(c) visual display means for displaying glucose levels and interfacing with the patient;
(d) voice processing means for processing the glucose level and synthesizing an auditory output that conforms with the glucose level, wherein said voice processing means is bidirectional and receives additional data from the patient and via a keypad concurrently with the glucose level then tested, including:
(1) dietary consumption;
(2) exercise;
(3) medical information (size, BMI, other conditions, other medications); and
(4) comments;
(e) storage means for storing said test data, said glucose level and said additional data linked to the patient for later retrieval;
(f) input/output means for transmitting said test data, said glucose level and said additional data to a treating physician and receiving further instructions as determined by the treating physician after the determination of said glucose level; and
(g) atomic clock means for generating real time clock signals from time code signals transmitted by a time standard and received by an antenna

6. The device of claim 5, wherein the further instructions are provided to the patient by output via said voice processing means.
